(19) United States
(12) Patent Application Publication
(10) Pub. No.: US 2003/0036556 A1
(43) Pub. Date: Feb. 20, 2003

(54) Zonisamide use in headache

(76) Inventor: Julianne E. Jennings, San Diego, CA (US)

Correspondence Address: HOWREY SIMON ARNOLD & WHITE, LLP, BOX 34, 301 RAVENSWOOD AVE., MENLO PARK, CA, 94025, US

(21) Appl. No.: 10/186,838

(22) Filed: Jun. 28, 2002

Related U.S. Application Data
(60) Provisional application No. 60/302,149, filed on Jun. 29, 2001.

(51) Int. Cl. A61K 31/42

(52) U.S. Cl. 514/379

(57) ABSTRACT

The present invention is directed to a method of treating headache, including migraine. The method comprises administering to a subject a pharmaceutical composition comprising an effective amount of zonisamide. The methods of the present invention are useful in relieving headache. The compounds of the present method can also be used in conjunction with other therapeutic agents commonly used to treat headache thus enhancing the therapeutic effect of reducing headache.

### FIELD OF THE INVENTION

The present invention relates to methods of treating headache, particularly migraine headache, with zonisamide (1,2-benzioxazole-3-methanesulfonamide).

### BACKGROUND OF THE INVENTION

Migraine headache (“migraine”) is a common disorder, believed to afflict 20 to 30 percent of the population, some transiently, some chronically. In migraine patients, throbbing head pain occurs at intervals. The pain often is associated with symptoms such as nausea, vomiting and impaired vision.

The biochemical mechanisms underlying migraine are uncertain. The predominate belief expressed in the literature for many years has been that vasodilation of extracranial vessels causes migraine. Treatment efforts, therefore, were aimed at methods of causing vasoconstriction. More recently, evidence has shown that activation of prejunctional 5-HT₁ heteroreceptors on primary afferent trigeminovascular fibers, by drugs such as ergot alkaloids and sumatriptan, alleviate migraine pain, suggesting a neuronal pathogenesis as opposed to a vascular one. Trigeminovascular fibers innervate meningeal blood vessels. The interaction of these compounds with the 5-HT1 receptor is very specific. These compounds do not interact with other 5-HT receptors, norepinephrine receptors, glutamate receptors or GABA (gamma-aminobutyric acid) receptors (Moskowitz, et. al., Annu. Rev. Med. 44:145-54 (1993)).

Several other studies suggest that a neuronal mechanism is involved in migraine. Clinical studies have reported the usefulness of valproic acid (2-propylpentanoic acid) for the prophylactic treatment of migraine. (Herring, et al., Cephalalgia, 12:81-84 (1992)), chronic daily headache (Mathew, Headache, 31:71-74 (1991)), and cluster headache (Herring, et al., Cephalalgia, 9:195-198 (1989)). Valproic acid, commonly used for the treatment of epilepsy, is a GABA transminase inhibitor and an activator of glutamic acid decarboxylase (Loscher, Journal of Neurochemistry, 36:1521-7 (1981)). Following its administration, GABA levels increase. It was postulated that sodium valproate could be exerting a GABA-mimetic effect by acting on GABA receptors, including those on the dorsal raphe nuclei, resulting in a decreased firing rate of the seratonergic neurons with a vasodilating effect and therefore preventing migraine by inhibiting vasodilation. (Jensen, et al., Neurology 44:647-651(1994)). It however has never been established that sodium valproate mediates its effect via GABA receptors, although the prevailing theory remains that vasodilation is central to migraine.

The above studies as well as others have resulted in the development of several therapeutic approaches for treating migraine; no one mechanism has been identified yet which appears to be responsible for migraine. A handful of drug treatments for migraine has been approved by the FDA: Inderal® (propanolol, Wyeth Ayerst), Sansert® (methysergide maleate, Novartis), Depakote® (divalproex sodium, Abbott), Blocarden® (timolol, Merck) and Imitrex® (sumatriptan, Glaxo Wellcome). None of the drugs tested thus far has been completely effective or free of side effects. Some side effects result from administering the drug in such high doses that toxicity rises to unacceptable levels. A real need exists to develop a class of drugs, which is very effective in treating migraine-type headache, but does not cause significant side effects.

The typical chronic daily headache, transformed migraine, can be associated with substance overuse. The typical transformed migraine begins with episodic migraine that insidiously becomes more frequent over time. Correspondingly, use of analgesics and other substances gains frequency until it becomes daily or nearly daily. This frequent use of palliative drug traps the patient in a rebound headache phenomenon. In this situation, a substance is used more than 3 days a week and its withdrawal triggers a headache that can only be treated by redosing the offending substance. This phenomenon has been reported with caffeine, simple analgesics, combination analgesics containing barbiturates, sedatives, or narcotics, ergots (e.g., ergotamine tartrate), and sumatriptan succinate and other serotonin agonists. It may also occur with decongestants. The problem is compounded by a tendency of substance overuse to make headaches refractory to otherwise effective prophylactic medications. The possible neurophysiologic mechanisms for chronic daily headache include kindling (a phenomenon similar to kindling in epilepsy), activation of nociceptive facilitatory systems, lack of inhibitory modulation, and suppression or down-regulation of an already suppressed or abnormal antinociceptive system. (Evans, R. W., Mathew, N. T., Handbook of Headache, Philadelphia: Lippincott, Williams & Wilkins, pp. 74-75 (2000). A clinical approach to treat this form of transformed migraine is to wean the patient away from the analgesics and then to institute treatment with other, unrelated, classes of migraine or headache medication.

There is a need for a new treatment modality for migraine and associated headache syndromes such as chronic daily headache and transformed migraine.

Zonisamide is an antiseizure drug classified as a sulfonamide and chemically unrelated to other antiseizure agents. Zonisamide has the chemical structure of 1,2-benzisoxazole-3-methanesulfonamide and is further characterized in the Merck Index (11^{th} Ed. 1989) at monograph no. 10094. Zonisamide and related structures are described in described in U.S. Pat. No. 4,172,896, which is hereby incorporated herein by reference in its entirety for all purposes. It is approved for use in humans in the United States and in Japan. The mechanism(s) by which zonisamide exerts its antiseizure activity is unknown. It has demonstrated anticonvulsant activity in threshold for generalized seizures in the kindled rat model and reduced the duration of cortical focal seizures induced by electrical stimulation of the visual cortex in cats. Furthermore, zonisamide suppressed both interictal spikes and the secondarily generalized seizures produced by cortical application of tungstic acid gel in rats or by cortical freezing in cats.

Zonisamide may produce anti-epileptic and anti-convulsant effects through action at sodium and calcium channels. In vitro pharmacological studies suggest that zonisamide blocks sodium channels and reduces voltage-dependent, transient inward currents (T-type Ca²⁺ currents), consequently stabilizing neuronal membranes and suppressing neuronal hypersynchronization. In vitro binding studies have demonstrated that zonisamide binds to the GABA/benzodiazepine receptor ionophore complex in an allosteric fashion which does not produce changes in chloride flux. Other in vitro studies have demonstrated that zonisamide (10-30 μg/mL) suppresses synaptically-driven electrical activity without affecting post-synaptic GABA or glutamate responses (cultured mouse spinal cord neurons) or neuronal or glial uptake of [³H]-GABA (rat hippocampal slices). Thus, zonisamide does not appear to potentiate the synaptic activity of GABA. In vivo microdialysis studies demonstrated that zonisamide facilitates both dopaminergic and serotonergic neurotransmission. Based on the effects such as blocking sodium channels and reducing voltage-dependent, transient inward currents (T-type Ca²⁺ currents), modulation of serotonergic and dopaminergic neurotransmission, Applicants have discovered that zonisamide is efficacious in treating headache of the migraine or chronic daily types.

### SUMMARY OF THE INVENTION

The present invention is directed to a method of treating headache in a subject in need of such treatment. The method comprises administering to a subject a pharmaceutical composition comprising zonisamide, in an amount effective to relieve headache. The invention provides a method for the treatment of headache pain, more particularly migraine, transformed migraine and chronic daily headache in mammals.

The pharmaceutical composition can be administered in the range of 50 mg to 600 mg per day through a variety of routes of administration, including oral, topical, rectal, injection, or implantation. A preferred route of administration is via oral dosing.

### DETAILED DESCRIPTION OF THE INVENTION

Zonisamide has a unique combination of pharmacologic actions: it inhibits voltage-gated sodium channels and also blocks T-type calcium channels. Applicants believe that these mechanisms play a role in headache modulation, via neuronal stabilization. The pharmacokinetic and drug interaction profiles of zonisamide are ideal for treating patients with headaches.

The present invention provides a method of treating a subject who inflicts headaches. The method comprises administering to the subject a pharmaceutical composition comprising an effective amount of zonisamide to relieve headaches.

Chronic daily headache (CDH) consists of two main divisions, long-lasting headaches and short-lasting headaches, each comprises the following clinical subtypes. Long-lasting headaches (i.e., attack duration longer than 4 hours) include transformed migraine (TM), chronic tension-type headache, new daily persistent headache, hemicrania continua, and analgesic round headache. Short-lasting headaches (i.e., attack duration less than 4 hours) include chronic cluster headache, chronic paroxysmal hemicrania, hypnic headache, and idiopathic stabbing headache.

A common chronic daily headache form is transformed migraine (TM). Patients with CDH are often classified with chronic tension-type headache. However, headaches in these patients frequently begin as typical episodic migraine attacks then evolve into a pattern of CDH. Therefore, categorizing such individuals as suffering from a type of tension headache seems inappropriate. (Silberstein, et al., (Headache 34:1-7 (1994)). A new classification, transformed migraine, better describes this type of headache. (Mathew, Cephalalgia 13 (suppl 12):78-83 (1993)).

When associated with overuse of analgesic medications, TM requires carefully monitoring substance withdrawal. Such substance withdrawal or detoxification is often accompanied by or followed by appropriate migraine abortives and long-term prophylaxis; management options include inpatient and outpatient protocols.

The compounds of the present invention can be applied by any of the accepted modes of systemic administration for agents which affect the central nervous system (CNS) including oral, parenteral, rectal, and otherwise systemic routes of administration. Any pharmaceutically acceptable mode of administration can be used, including solid, semi-solid, or liquid dosage forms, such as for example, tablets, suppositories, pills, capsules, powders, liquids suspensions, or the like, preferably in unit dosage form suitable to single administration of precise dosages, or in sustained or controlled release forms for the prolonged administration of the compound at a predetermined rate. The compositions typically include a conventional pharmaceutical carrier or excipient and the drug product zonisamide and, in addition, can include other medicinal agents, pharmaceutical agents, carriers, etc. The compositions are advantageously compounded into unit dosage forms, containing a predetermined, standard amount of the active compound, to make dosing and patient compliance simpler.

The amount of active compound administered depends on the subject being treated, the severity of the affliction, the manner of administration and the judgment of the prescribing physician. However, an effective dosage is in general in the range of 50 to 600 mg/day, preferably 100-300 mg/day, which may be administered all at a time or in divided doses. The dosage of these compounds may vary in accordance with the administration route, the age of the patient and the degree of the therapeutic effect desired.

The compounds of the present invention are usually administered in the form of a pharmaceutical composition that contains them in admixture with a pharmaceutical carrier. The pharmaceutical composition can be in the dosage forms such as tablets, capsules, granules, fine granules, powders, syrups, suppositories, injections, or the like. These preparations can be prepared by conventional methods.

The carriers useful for these preparations include all organic or inorganic carrier materials that are usually used for the pharmaceutical preparations and are inert to the active ingredient. Examples of the carriers suitable for the preparation of tablets capsules, granules and fine granules are diluents such as lactose, starch, sucrose, D-mannitol, calcium sulfate, or microcrystalline cellulose; disintegrators such as sodium carboxymethylcellulose, modified starch, or calcium carboxymethylcellulose; binders such as methylcellulose, gelatin, acacia, ethylcellulose, hydroxypropylcellulose, or polyvinylpyrrolidone; lubricants such as light anhydrous silicic acid, magnesium stearate, talc, or hydrogenated oil; or the like. When formed into tablets, they may be coated in a conventional manner by using the conventional coating agents such as calcium phosphate, carnauba wax, hydroxypropyl methylcellulose, macrogol, hydroxypropyl methylphthalate, cellulose acetate phthalate, titanium dioxide, sorbitan fatty acid ester, or the like.

Examples of the carriers suitable for the preparation of syrups are sweetening agents such as sucrose, glucose, fructose, or D-sorbitol; suspending agents such as acacia, tragacanth, sodium carboxymethylcellulose, methylcellulose, sodium alginate, microcrystalline cellulose, or veegum; dispersing agents such as sorbitan fatty acid ester, sodium lauryl sulfate, or polysorbate 80; or the like. When formed into syrups, the conventional flavoring agents, aromatic substances, preservatives, or the like may optionally be added thereto. The syrups may be in the form of dry syrup that is dissolved or suspended before use.

Examples of bases used for the preparation of suppositories are cacao butter, glycerin saturated fatty acid ester, glycerogelatin, macrogol, or the like. When formed into suppositories, the conventional surface active agents, preservatives or the like may optionally be admixed.

When formed into injections, the compound is dissolved in distilled water for injection, to which may optionally be added the conventional solubilizers, buffering or pH adjusting agents, isotonic agents, preservatives and other suitable substances. The injections can be in the solid dry preparations, which are dissolved before use.

These pharmaceutical compositions usually contain zonisamide or as the active ingredient in an amount of 0.5% by weight or more, preferably 10 to 70% by weight, based on the total weight of the composition. These compositions may optionally contain other therapeutically active compounds.

For solid compositions, conventional non-toxic carriers include, for example mannitol, lactose, starch, magnesium stearate, sodium saccharin, talcum, cellulose, glucose, sucrose, magnesium carbonate, and the like may be used. The active compound as defined above may be formulated as suppositories using, for example, polyalkylene glycols, for example, propylene glycol as a carrier. Liquid pharmaceutically administerable compositions can, for example, be prepared by dissolving, dispersing, etc. an active compound as defined above and optional pharmaceutical adjuvants in a carrier, such as, for example, water, saline, aqueous dextrose, glycerol, ethanol, and the like to thereby form a solution or suspension. If desired, the pharmaceutical composition to be administered may also contain minor amounts of non-toxic auxiliary pH buffering agents and the like, for example, sodium acetate, sorbitan monolaurate, triethanolamine oleate, etc. Actual methods of preparing such dosage forms are known, or will be apparent to those skilled in this art; for example, see Remington's Pharmaceutical Sciences, Mack Publishing Company, Easton, Pa., 15th Edition, 1975. The composition or formulation to be administered will, in any event, contain a quantity of the active compound in an amount effective to alleviate the symptoms of the subject being treated.

Dosage forms or compositions containing active ingredient in the range of 0.25 to 95% with the balance made up from non-toxic carrier may be prepared. For oral administration, a pharmaceutically acceptable non-toxic composition is formed by the incorporation of any of the normally employed excipients, and may contain 1%-95% active ingredient, preferably 5%-50%.

Parenteral administration is generally characterized by injection, whether subcutaneously, intramuscularly, or perineurally. Injectables can be prepared in conventional forms, either as liquid solutions, suspensions, or emulsions. Suitable excipients include, for example, water, saline, aqueous dextrose, glycerol, ethanol or the like. In addition, the pharmaceutical compositions may also contain minor amounts of non-toxic substances such as wetting or emulsifying agents, auxiliary pH buffering agents and the like, such as, sodium acetate, sorbitan monolaurate, triethanolamine oleate, etc.

The percentage of active compound contained in such parenteral compositions is highly dependent on the specific nature thereof, as well as the activity of the compound and the needs of the subject. However, percentages of active ingredient in solution are in general 0.1% to 10%, and preferably 0.2-2%.

Other modes of administration can also be practiced in accordance with the present invention. For example, intravenous, intramuscular, and subcutaneous delivery are examples of delivery methods that are contemplated by the present invention.

For delayed release, the compounds of the invention may be formulated in a pharmaceutical composition, such as in microcapsules formed from biocompatible polymers, or in liposomal carrier systems according to methods known in the art.

For continuous release of active agent, the compound may be covalently conjugated to a water soluble polymer, such as a polylactide or biodegradable hydrogel derived from an amphipathic block copolymer, as described in U.S. Pat. No. 5,320,840. Collagen-based matrix implants, such as described in U.S. Pat. No. 5,024,841, are also useful for sustained delivery of therapeutics.

The method of the present invention can be used with other therapeutic agents commonly used to treat headache, thus enhancing the effects of therapeutic agents and adjunctive agents. Other therapeutic agents used include Inderal® (propanolol, Wyeth Ayerst), Sansert® (methysergide maleate, Novartis), Depakote® (divalproex sodium, Abbott) and Blocarden® (timolol, Merck) and Imitrex® (sumatriptan, Glaxo Wellcome).

High doses are sometimes required for some therapeutic agents to achieve levels to effectuate the target response, but high doses often associate with a greater frequency of dose-related adverse effects. Thus, combined use of the pharmaceutical composition of the present invention with therapeutic agents commonly used to treat headache allows the use of relatively lower doses of other agents, which results in a lower frequency of adverse side effects associated with long-term administration of such agents. Thus, another advantage of the compounds in this invention is to reduce adverse side effects of drugs used to treat headache, such as tolerance, dependence, constipation, respiratory depression, sedation, and gastrointestinal side effects.

The following examples further illustrate the present invention. These examples are intended merely to be illustrative of the present invention and are not to be construed as being limiting.

### EXAMPLES

### Example 1

Methods:

Thirty-three (33) patients from an active headache clinic population were selected for open-label treatment with zonisamide. All patients had refractory migraines and mixed headache disorders, as defined by International Headache Society criteria. All had failed or responded poorly to trials with other anticonvulsants as prophylactic therapy. Most patients (n=27) had failed at least 2 such agents, and 17 had failed 3 agents. Eight patients had chronic migraines alone, with a mean of 9 migraines per month, and 25 had tension-type headache (TTH) with a mean of 7.7 migraines per month. (Table 1). Zonisamide was initiated as adjunctive therapy: 100 mg of the medication was given in the evening or at bedtime every fourth day for 4 to 5 doses.

TABLE 1
Patient Headache Characteristics

| | | Mean headache frequency |
|---|---|---|
| Headache Type | n | (episodes/month) |
| Chronic migraine | 8 | 9 |
| Tension-type with migraine | 25 | 7.7 |

Consequently, dosing was increased to every third day, every other day, and finally to a daily dose. Dose adjustments of zonisamide were made in increments of 100 mg, every 2 to 3 weeks, with a daily maximum dose of 500 mg, in some cases. Headache severity was rated on a 0 to 10 numeric rating scale (NRS).

Results

After at least 2 months of zonisamide therapy, 6 patients have reported a 65% or better decrease in frequency of headaches; 8 reported a 25% to 50% decrease in headaches (Table 2). Nine patients (27%) discontinued zonisamide therapy; of these, 3 (9% of total) did not respond, (6% of total) were noncompliant, and 4 (12% of total) dropped the medication due to side effects. Ten patients have just been started on the medication and are in the titration phase of therapy.

TABLE 2
Results of Zonisamide Treatment*

| | n |
|---|---|
| ≧65% decrease in headache frequency | 6 |
| 25% to 50% decrease in headache frequency | 8 |
| No response | 3 |
| Noncompliance | 2 |
| Discontinuation due to side effects | 4 |

*Note:
10 patients were still in the zonisamide titration phase of therapy at the time the data was collected.

Conclusions:

This open-label study, which is one of the first to investigate zonisamide for treating refractory headache disorders, suggests that zonisamide has efficacy in a very difficult-to-treat refractory headache population. These data suggest that zonisamide, like virtually all other neuronal stabilizing agents, has utility where older agents have failed to provide relief.

### Example 2

Sixteen patients who received zonisamide for prophylaxis of CDH (i.e., >15 headache days/month) for at least 3 months were included in this study. All patients had previously failed treatment with at least 2 prophylactic medications (mean=5.9, range=2 to 10). Zonisamide therapy was initiated at 100 mg/day, and 6 patients were titrated to a zonisamide dosage of 200 mg/day after 2 weeks. Patient headache diaries were used to record information, regarding headache frequency, duration, severity, and disability ratings. Headache severity and disability were rated using a 4-point scale (0=none; 1=mild; 2=moderate; 3=severe). Headache diaries and clinic records were used to determine mean headache duration, frequency, severity, disability for each patient 1 month prior to initiation of zonisamide therapy (baseline) and again 3 months after stable zonisamide dosage was established.

Results:

All patients (n=16) were female (age mean=37.3, range=20 to 57). Prior to initiation of zonisamide therapy, patients experienced a mean of 22-headache days/month. Mean headache duration was 8.46 hours. After 3 months of zonisamide treatment (mean dosage=137.5 mg/d), the mean number of headache days/month was reduced by 34% to 5 days, and the mean headache duration was reduced by 24% to 6.41 hours (Table 1). Total headache time was reduced 50% (186 hours/month at baseline, 93 hours/month on zonisamide). Mean headache rating decreased 23% (1.84 at baseline, 1.41 on zonisamide), and mean disability rating decreased by 24% (1.48 at baseline, 1.12 on zonisamide) (Table 3). Reports of adverse events included mild diarrhea in 2 patients and weight loss (mean=11 lb.) in 9 patients.

TABLE 3
Results of Zonisamide Treatment

| | Baseline | Zonismide* |
|---|---|---|
| Mean number of headache days per month | 22 | 14.5 |
| Mean headache duration (hours) | 8.46 | 6.41 |
| Mean headache severity rating | 1.84 | 1.41 |
| Mean disability rating | 1.48 | 1.12 |

*After 3 months of zonisamide therapy at a stable dosage, mean dosage = 137.5 mg/d

Conclusions:

This study suggests that zonisamide has clinical utility in patients with CDH who have been refractory to numerous prophylactic medications. Notably, total headache time was reduced by 50% after 3 months of therapy at a stable zonisamide dosage. Zonisamide treatment was well tolerated by patients in this study.

### Example 3

Methods:

Twenty-five patients with various chronic pain disorders were selected for treatment with zonisamide. All had neuropathic pain of one kind or another. Fifteen had failed back/neck surgeries with ongoing radicular pain, 4 had complex regional pain syndrome (type 1), 4 had diabetic neuropathy, and 2 had painful sensory neuropathy. All had failed, or responded minimally at best, to trials of numerous other anticonvulsants (neuronal stabilizing agents) for prior treatment of pain symptoms. Eighteen were on such therapy at the time of starting zonisamide therapy. Twenty-two patients with refractory migraine headaches of mixed headache disorders were similarly selected for zonisamide therapy. Most (n=17) had failed 3 or more other neuronal stabilizing agents as prophylaxis therapy for their headaches. Five patients had chronic migraines alone, with an average of 9 migraines per month. Zonisamide was begun at 100 mg every third day for 3 to 5 doses, with an increase to every-other-day dosing and then to a daily dose. Dosage changes were made every 2 weeks at a minimum. Headache and pain spores were rated on a 0 to 10 numeric rating scale by each patient.

Results:

Pain reductions averaged 60% or better for 6 patients. Six additional patients reported reductions in pain of 30% to 50%. Four patients experienced slight reductions (less than 25%), and 7 patients had no pain relief and/or side effects. Two patients were lost to follow-up or discontinued the medication prematurely.

Conclusions

Zonisamide has efficacy in a difficult-to-treat population of pain and headache patients. The results suggest that zonisamide is a potent therapeutic addition in refractory cases of pain or headache.

### Example 4

Methods:

This study included 30 patients with various chronic pain disorders, all with neuropathic pain (Table 1). All patients had previously failed or responded minimally to numerous other anticonvulsants (neuronal stabilizing agents) for treatment of painful symptoms. Twenty-three patients were on such therapy at the time zonisamide therapy was initiated.

TABLE 1
Types of Pain Experienced by Study Subjects

| N | Type of Pain |
|---|---|
| 26 | Mixed headache syndrome |
| 20 | Lumber or cervical radiculopathy due to disc disease or failed |
| | back/neck surgery with ongoing radicular pain |
| 8 | Chronic migraine |
| 4 | Complex regional pain syndrome (type 1) |
| 4 | Diabetic neuropathy |
| 2 | Painful sensory neuropathy |

Zonisamide therapy was initiated at 100 mg every fourth day for 3 to 6 doses, with an increase to every third day, then to every other day, and finally to a daily dose. Adjustments in the dose of zonisamide were made no more than every 2 weeks. Headache and pain scores were rated on a 0 to 10 numeric rating scale (NRS) by each patient.

Results:

Reductions in pain scores reported by chronic pain patients while taking zonisamide are summarized in FIG. 1. After at least 2 months of zonisamide therapy, 10 patients (30%) reported pain reductions of 60% or greater, and 7 patients (23%) reported 30% to 50% reductions in pain. Five patients (17%) experienced slight (i.e., less than 25%) pain reductions, and 5 patients (17%) experienced no pain relief and/or reported side effects while receiving zonisamide. Three patients (10%) wee lost to follow-up or discontinued the medication prematurely.

The invention, and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as invention, the following claims conclude the specification.

What is claimed is:

1. A method of treating headache in a subject in need of such treatment, said method comprising:
administering to a subject a pharmaceutical composition comprising zonisamide, in an amount effective to relieve headache.

2. The method according to claim 1, wherein said headache is long-lasting headache.

3. The method according to claim 2, wherein said long-lasting headache is transformed migraine headache.

4. The method according to claim 1, wherein said headache is short-lasting headache.

5. The method according to claim 1, wherein said effective amount of zonisamide is in the range of about 50 to about 600 mg/day.

6. The method according to claim 5, wherein said effective amount of zonisamide is in the range of about 100 to about 300 mg/day.

7. The method according to claim 1, wherein said pharmaceutical composition is administered orally to said subject.

8. The method according to claim 1, wherein said pharmaceutical composition is administered topically to said subject.

9. The method according to claim 1, wherein said pharmaceutical composition is administered in combination with another therapeutic agent commonly used to treat headache.
